(19) United States
(12) Patent Application Publication
(10) Pub. No.: US 2012/0108858 A1
(43) Pub. Date: May 3, 2012

(54) METHOD AND DEVICE FOR MANUFACTURING OF RADIOPHARMACEUTICALS

(76) Inventor: MAXIM KISELEV, BROADLANDS, VA (US)

(21) Appl. No.: 12/982,499

(22) Filed: Dec. 30, 2010

Related U.S. Application Data
(60) Provisional application No. 61/389,448, filed on Oct. 28, 2010.

(51) Int. Cl. C07C 25/00

(52) U.S. Cl. 570/123

(57) ABSTRACT

The invention relates to a device for processing radioisotopes and production of radiopharmaceuticals, for example, 18F-2 Deoxy-2-Fluoro-D-Glucose (FDG), for Positron Emission Tomography (PET). Use of a tilting and rotating reactor with optimized geometry allows for decreased processing time and increased efficiency. Reagent vials are separated from the reactor module and placed in a separate enclosure allowing safe reloading of the system and production of multiple batches with minimal operator exposure to radiation. Reagent vials enclosure is protected from the environment and filled with purified air to reduce risk of contamination. Product and reagents pathways are sealed from the environment and are sterilized.

### CROSS-REFERENCE TO RELATED APPLICATIONS

This application claims priority from U.S. provisional application No. 61/389,448 by the same inventor, specification filed on Oct. 4, 2010, drawings filed on Oct. 28, 2010, all being incorporated herein by reference in their entirety

### BACKGROUND

1. Technical Field

The invention relates to methods and apparatus for processing radioisotopes to manufacture radiopharmaceutical preparations used for diagnosis of cancer, more particularly processing of a cyclotron-produced F-18 isotope and production of F-18 labeled compounds such as F-18-2 Deoxy-2-Fluoro-D-Glucose (FDG).

2. Background

FDG is used for cancer diagnosis by Positron Emission Tomography (PET). In the course of PET studies, a small quantity (10-15 mCi) of FDG is typically injected intravenously into a patient enabling visualization of the distribution of radioactive material which is known to accumulate within malignant tumors. Due to the short half-life time of the F-18 isotope (109 min), it is necessary to manufacture such products in a large quantity, on the order of 100-1000 times greater than the quantity actually used, to allow for decay in transit prior to use. Although the F-18 isotope can be produced in relatively large amounts, up to 50,000 mCi/batch, efficient and safe processing of such a high quantity presents certain challenges, such as autoradiolytic decomposition of the product and risk of contamination, in particular, microbial contamination of the product and its ingredients. Another challenge is to minimize operator radiation exposure.

Two methods of FDG production are known, see, for example, Fowler et al. (2002) for a review. The first method, discovered in 1970's involves electrophilic fluorination of 3,4,6-triacetilglucal (TAG) with a suitable fluorination agent. This method requires, among other steps, evaporation of a large volume of an organic solvent. Due to its complexity, low yield, and poor product quality this method was abandoned in the mid-1980's when a nucleophilic method involving substitution of a triflate group in 1,3,4,6-tetra-O-acetyl-2-trifluoromethanesulfonyl-D-mannose with F-18 fluoride was introduced. This currently preferred nucleophilic method no longer requires evaporation of a large quantity of organic solvents after hydrolysis and produces FDG without isomeric impurities and in a relatively short time.

A number of automated methods and systems for FDG production using the nucleophilic method have been developed. Examples of such systems can be found in U.S. Pat. No. 4,794,178 to Coenen et al. and U.S. Pat. No. 6,599,484 to Zigler et al., incorporated herein by reference. All previously described systems utilize one or several reactor vessels which remain essentially stationary during introduction of reagents, synthesis, and removal of the product. Such reaction vessels are typically cylindrical in shape and are positioned vertically. They may have flat or coned (V-shaped) bottom. One such system, depicted in FIG. 1 of U.S. Pat. No. 5,932,178 to Yamazaki et al., appears to utilize an inclined reactor vessel having a bottom which is the apex of a cone having sides that are reasonably straight for some distance to a shoulder which then narrows to a neck. The term “cone-bottom pear-shaped vessel” will be used herein to describe this shape. Although use of such vessels in organic chemistry and in radiochemistry is known, no references describe use of a rotating cone-bottom pear-shaped vessel for making FDG.

Although all reactors previously used for automated FDG production are essentially stationary, in general, use of rotating and tilting flasks in chemistry is known. In particular, rotary evaporators are commonly used in laboratory practice for evaporating solvents. Typical examples of such devices are found in U.S. Pat. No. 4,759,825 to Medvey et al., U.S. Pat. No. 4,780,178 to Yoshida et al., and U.S. Pat. No. 6,740,206 to Genser, all incorporated herein by reference. They are typically equipped with round-bottom or pear-shaped round-bottom rotating evaporation flasks such as the item 11 depicted in FIG. 3 Yoshida et al.

There are two references, Iwata et al. (1984) and Alexoff et al. (1986) disclosing the use of rotary evaporators in manual synthesis of FDG via the electrophilic method for the purpose of evaporating solvents after hydrolysis. In both cases, the authors conclude that these devices are unsuitable for automated synthesis and no attempt to use a rotating vessel as a reactor for automated FDG synthesis was disclosed.

A common method of making FDG is to use a single use “kit” of components which typically includes various reagent and processing vessels, tubes, and valves etc., and is pre-sterilized by gamma radiation before use. Examples of such systems are provided in U.S. Pat. Nos. 5,312,592 and 5,415,843 to Andersson, U.S. Pat. No. 6,172,207 to Damhaut et al., U.S. Pat. App. 2004/0028573 by Schmitz et al., and U.S. Pat. No. 7,235,216 to Kiselev et al., all of these being incorporated herein by reference. The kit is typically designed to work in a combination with a stationary reusable apparatus, which includes gas and vacuum supply lines, connections for introducing some reagents and removing reaction products, and other parts. The stationary apparatus is not sterilized. The disposable kit is assembled before use and attached to a part of the apparatus immediately before processing of each batch. Afterwards, the now partially radioactive kit is ejected or removed manually and placed in a shielded container. Schmitz et al. review prior art kits and conclude that some are too costly for single use, see paragraphs [0009] and [0010]. However, as noted by Kiselev et al. at col. 8, 11. 62-64, the advantage is that the entire kit can be preloaded with reagents and an automated process accomplished without extensive preparation. Another advantage is, see col. 2, 1. 1, cross-contamination between batches is reduced. To reduce cross-contamination between batches, both Schmitz et al. and Kiselev et al. include in their kits, not only reagent vials, but reactor vessels and associated connecting tubing, see their FIG. 1 and FIG. 2, respectively. Currently, it is thought this method of processing is utilized in over 80% of all FDG production facilities.

FIG. 1a shows a schematic representation of a typical prior art FDG synthesizer placed inside lead shielded enclosure 3. Radioactive material, typically F-18 fluoride is delivered via tube 1 directed through a shielded conduit 2, while all other reagents in quantity sufficient to synthesize one batch of FDG are placed in containers 6 which are attached to a disposable kit or “cassette” 5 together with reactor 9a and various tubes, valves and filters that may be required for automated processing. The kit 5 is attached to stationary apparatus 4 which is also connected to a waste bottle 10 and product container 7. The later is typically placed within a transportable shield 8. The kit 5 has to be replaced prior to production of each batch of FDG. In older systems, this method required extensive manual manipulations within shielded enclosure 3 prior to production of each batch of FDG, but newer systems have reduced the amount through more automation. In any case, the method allows reduction of the bioburden by sterilization of the disposable kit prior to use by gamma radiation, although there is usually no provision for sterilizing adjacent parts of the stationary apparatus 4.

Another, less common, prior art arraignment for FDG processing involves a stationary apparatus without a disposable kit as shown in FIG. 1b, wherein like numerals are used for like parts as in FIG. 1a. This type of apparatus is described, for example in the “Manual and Operating Instructions” Nuclear Interface Datentechnik für Strahlungsmeβgeräte GmbH, Oct. 27, 2000, Munster, Germany. In this case, the reagents are typically placed in refillable containers 10 attached directly to the stationary apparatus 4 which also contains reactor 9a and all associated plumbing. This method does not allow for sterilization of the reactor and other parts of the system and typically requires manual manipulations within the shielded enclosure 3. A modification of this method, described in U.S. Pat. No. 7,718,436 to Zigler et al., utilizes reagent vessels pre-loaded with reagents sufficient to produce multiple batches of FDG. Besides saving the cost of a disposable kit, this eliminates the need to access the shielded enclosure 3 between batches.

Still a third arraignment in the prior art (not illustrated) is like FIG. 1b, but the reagent vessels 10 are outside the shielded enclosure 3 and feed in to reactor 9 via a tube through an opening in the shield. Examples can be found in laid-open Japanese patent applications by Suzuki et al. (JP2006-343289) and Tanaka et al. (JP2009-047454) and, shown generally, in Najafi, U.S. Pat. App. No. 2005/0265906. This arraignment has the advantage that no operator exposure to the inside of the shielded enclosure is needed to supply reagents either initially or to refill between batches and there is no disposable kit cost. Suzuki does make provision for feeding HEPA filtered air into both the reaction chamber and reagent enclosure.

Because FDG is generally administered intravenously, it must be sterile and pyrogen free which is usually achieved by filtration through a sterilizing 0.2 micron filter. Although sterilizing filtration is generally accepted by the FDA as an acceptable method for terminal sterilization of PET radiopharmaceuticals, it is known to have limited efficiency. Therefore, contamination of the final drug product prior to filtration must be kept to a minimum to reduce the bioburden on the filter. Prevention of contamination during processing is therefore a concern for PET radiopharmaceutical manufacturers. Due to this concern, the method of FDG production with use of disposable kits (FIG. 1a) is generally preferred because it enables a reduction of bioburden by gamma sterilization of the disposable components prior to use. However, this method requires manual operations with pre-sterilized components such as installation of the disposable kit 5 onto the stationary apparatus 4 prior to production of each batch of FDG. Furthermore, fluidic connections between the kit and stationary apparatus must be established to enable introduction of the radioactive materials and removal of the product and waste and thus, the integrity of the pre-sterilized kit must be violated within the environment of the shielded enclosure 3.

In general, manual manipulations with sterilized components are considered to be a likely source of error, create contamination risk, and should be avoided. The FDA recommends (Guidance for Industry document entitled “Sterile Drug Products Produced by Aseptic Processing Current Good Manufacturing Practice” (GMP) issued in 2004, on p. 10) that “The design of equipment used in aseptic processing should limit the number and complexity of aseptic interventions by personnel . . . . Rather than performing an aseptic connection, sterilizing the preassembled connection using sterilize-in-place (SIP) technology also can eliminate a significant aseptic manipulation.” Although the referenced document does not apply directly to PET radiopharmaceuticals, which are usually are not processed aseptically, the general principles of avoiding contamination are applicable.

Another problem facing developers of F-18 processing equipment is the radioactive nature of the product, specifically the beta-radiation burden on materials, reagents, solvents and the equipment itself. As a result of radiation-induced damage at a certain level of processed activity, typically over 5-10 Ci per batch or greater, the process efficiency of all existing production methods is known to decline. As much as 10% of the useful product or intermediate materials decompose during processing, thus reducing radiochemical yield. Lower yield reduces the amount of the product and increases its cost per dose, thus negatively affecting the overall cost of healthcare. A total of over 1.4 million doses of FDG are used in the U.S. each year with an average cost per dose of $245 (“2008 PET Imaging Market Summary Report”, December 2008, IMV, Des Plaines, Ill.). Loss of 10% of this product is equivalent to 34 million dollars per year.

### SUMMARY

There is described a method for automated synthesis of fluorine F18-labeled compounds utilizing a nucleophilic substitution reaction including the steps of: a) providing at least one reactor having a reactor axis, a mechanism to rotate the reactor about its axis, and a mechanism to tilt the reactor axis from substantially vertical to an inclined angle; and b) introducing at least one reactant into the reactor to perform synthesis and, during at least a part of the synthesis, tilting the reactor, so that a lower of the reactor sides is substantially horizontal, and rotating the reactor about its axis, so that the sides of the reactor are wetted by the reactants.

Not essentially, but possibly advantageously, a cone-bottom pear-shaped reactor can be used and the inclined angle is then approximately 50 degrees.

The method is particularly useful when the F-18 fluoride has an activity of greater than about 10 Ci per batch.

Typically, the method includes a drying step which is advantageously aided by a reactor rotation and a heating oil bath moved into position to surround the reactor during synthesis and moved away from the reactor after synthesis.

Final reaction products may be extracted by tilting the reactor to a substantially vertical position, inserting an extraction tube, extracting the products, and removing the extraction tube in preparation for a succeeding batch.

The apparatus for carrying out the above method comprises: a) at least one reactor having a reactor axis; b) a mechanism to rotate the reactor about its axis; and c) a mechanism to tilt the reactor axis. Advantageously, the reactor is cone-bottom pear-shaped and rotated with a motor and timing belt and can include a heating bath with a mechanism to move it into and out of a position surrounding the reactor and there is a mechanism to insert and retract a reaction-product extraction tube into an out of the reactor.

With the above method and apparatus, reactants are spread over a relatively large area to speed up any drying and, it is believed, reduce autoradiolytic decomposition.

Another aspect of the invention is a safe and sterile method for introducing reagents into the reactor including the steps of: a) providing a separate environmentally protected reagent enclosure placed outside of said shielded enclosure containing said reactor apparatus; b) opening said enclosure and manually placing at least one single-use sealed container containing at least one reagent into said reagent enclosure in a manner that said container seal remains intact until a further step; c) closing said reagent enclosure and purging with clean filtered air to remove contamination; and d) automatically penetrating said reagent container seal to cause the reagent to be deposited into said reactor apparatus by application of vacuum therein.

Additional steps may include sterilizing-in-place the reactor apparatus with a gaseous sterilant where the sterilant could be ozone, vaporized hydrogen peroxide, ethylene oxide, or some combination and/or also, in step “c” using ISO class 7 or better air.

The gaseous sterilant can be further directed into the reagent enclosure. Preferably, ozone or vaporized hydrogen peroxide can be used to sterilize the enclosure.

Lastly, monitoring the air may be used to ensure compliance with GMP.

With this method of placing reagent vessels an enclosure with their seals intact until the enclosure is closed and sterile environment is established inside, contamination risk is reduced. This is because, only after that and without manual intervention are the vessel seals broken and reagents transferred to the reactor.

### BRIEF DESCRIPTION OF THE DRAWINGS

FIG. 1a (prior art) is a schematic representation of a typical system with a disposable kit placed within a shielded enclosure containing a reactor module;

FIG. 1b (prior art) is a schematic representation of a typical system with refillable reagent containers placed within the shielded enclosure;

FIG. 2 is a schematic representation of a system comprised of the shielded reactor module and a separate reagent module outside the shielded enclosure;

FIG. 3 is a cross-sectional view of the reactor module represented in FIG. 2;

FIG. 4a is plan view of the reagent module represented in FIG. 2;

FIG. 4b is a sectional view of a single reagent dispenser assembly illustrated in FIG. 4a; and

FIG. 5 is a schematic representation of the processing and reagent modules illustrated in FIGS. 2, 3, 4a, & 4b.

### DETAILED DESCRIPTION

The following describes the best way of carrying out the invention. All specific materials, sizes, dimensions, suppliers and parts mentioned are provided to enable easy reproduction of the invention and are not limiting.

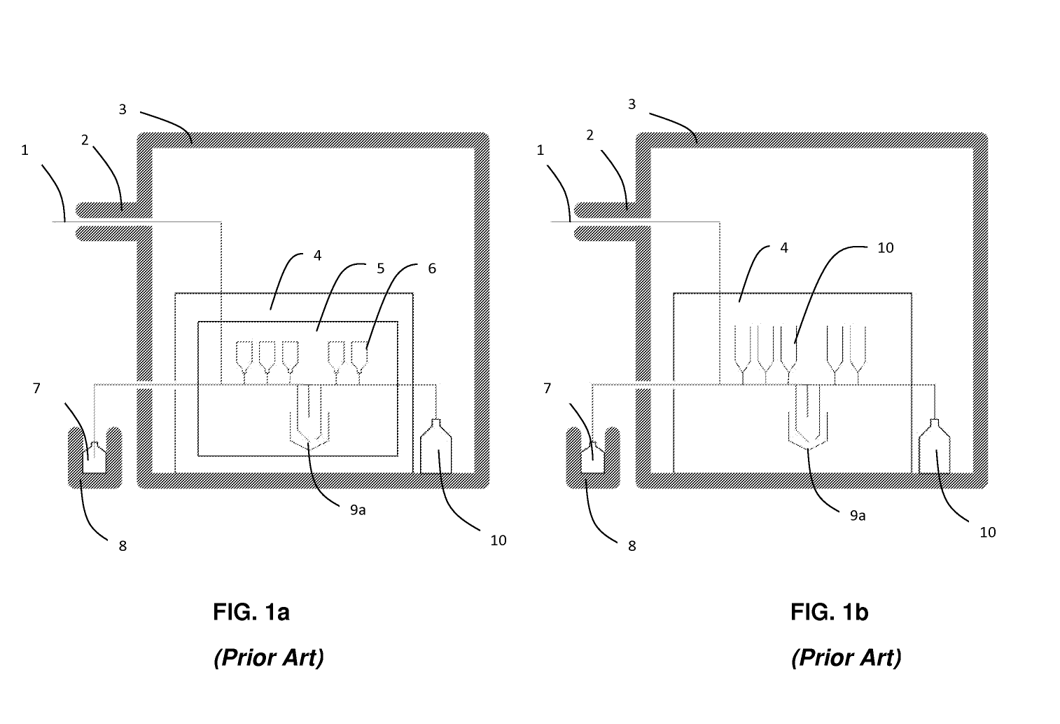

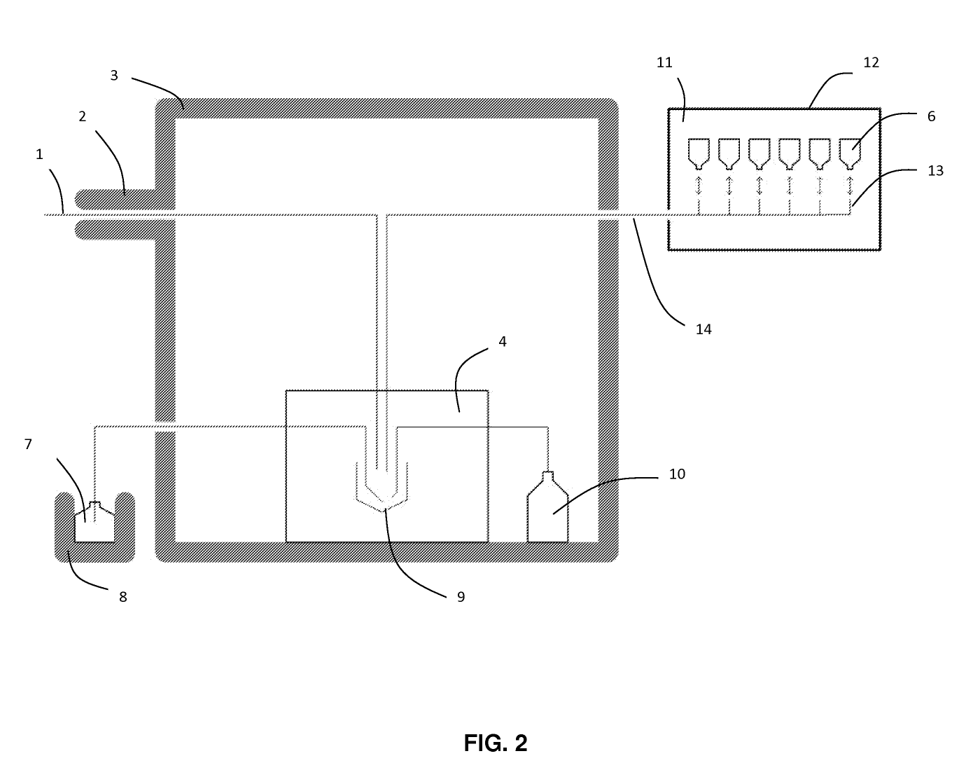

Referring to FIG. 2, using the same numerals where appropriate for similar functions as illustrated in FIGS. 1a and 1b, the system consists of two modules: a reactor module 4 and a separate reagent module 11. In general, the reactor module 4 is comprised of a reactor 9 along with tubes, valves, pumps and other components that come in contact with radioactive materials and is placed within a lead-shielded enclosure 3 to protect the operator from gamma radiation. The reagent module 11 contains a plurality of reagent vessels 6 (which, however, differ in detail from the prior art), associated valves, and mechanically actuated needles 13 and tubing, all contained in a clean-air enclosure 12 placed outside of the lead-shielded enclosure 3. In the course of automated processing, radioactive material, typically F-18 fluoride, is supplied via tube 1 placed within shielded conduit 2. During processing all radioactive material is confined to shielded enclosure 3 which also contains waste container 10 where radioactive waste is collected after processing. The useful product is delivered to a product container 7 placed within a separate shielded enclosure 8 which can be removed for future use. The reagent module enclosure 12, communicating with reactor 9 via tube 14, is placed outside the shielded enclosure where it may be conveniently adjoined with a user interface screen and other components. Separation of reagent storage vessels 6 from the reactor module 4 allows safe replacement of reagent vessels without exposure of the operator to radioactivity within shielded enclosure 3 and thus manufacturing of multiple batches of various products without excessive radiation exposure to the operator.

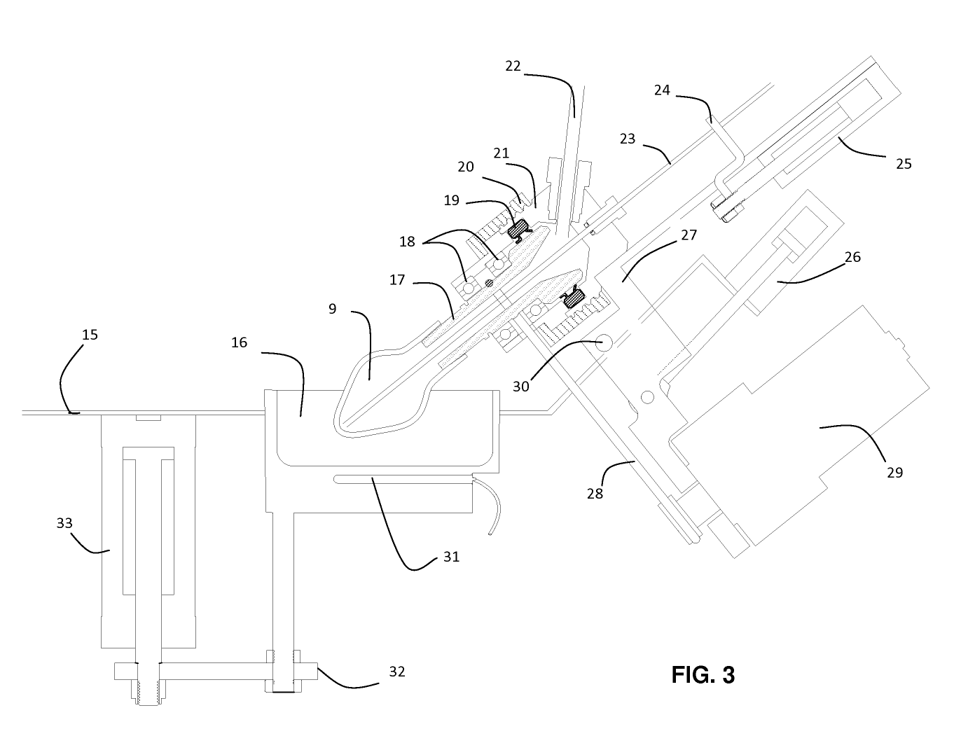

FIG. 3 shows a cross-sectional view of a part of the reactor module which includes a reactor, heater, and associated mechanical implements mounted on a supporting structure 15 fabricated from stainless steel. Commonly used standard components such as various valves, pumps, fittings and interconnecting tubes are not shown in this drawing for clarity. They are attached to the same support forming a processing unit which is placed within a lead-shielded enclosure. General principles of construction of radiochemical processing systems and use of commercially available valves, tubes, etc. are well known to those skilled in the art and taught in the prior art. Refer, for example, to U.S. Pat. No. 5,759,513 to Nakazawa, and U.S. Pat. No. 6,172,207 to Damhut et al., both of which are incorporated herein by reference.

Heating oil bath 16 is made of aluminum alloy and supported by elevator 32 which can be moved up and down by means of pneumatic cylinder 33. The bath is shown in its elevated position in the drawing. The bath 16 is equipped with one or more electric cartridge heaters 31 and can be heated up to 175° C. The bath can also be cooled by a stream of suitable coolant, such as cold air, supplied via separate tubes (not illustrated).

As illustrated, reactor 9 is a cone-bottom pear-shaped vessel, either a 10 or 25-ml flask, selected according to desired volume of the product and/or surface area spreading (explained below), with a standard JT14-10 threaded outer joint and screw cap (Minum-Ware® supplied by Chemglass Life Sciences, Vineland, N.J.). A variety of different shapes of similar flasks are also available and can be used. The reaction vessel is attached to the hollow center rotating shaft 17 fabricated from polyetheretherketone (PEEK) and supported by two ball bearings 18 made of polyformaldehyde (Delrin®) and having glass balls. The bearings are attached to reactor cap 21 by standard GL45 retaining nut 20.

The reactor is hermetically sealed by means of graphite filled Teflon Flexilip® seal 19 (Parker Seals, Lexington Ky.). Vacuum is applied to reactor 9 by means of ¼″ OD tube 22 made of silicone polymer (Tygon®, Saint-Gobain, France) and attached to reactor cap 21. Up to five additional tubes made of PEEK can also be attached to reactor cap 21 and fed through the hollow center in rotor 17. These tubes are used for introduction of various fluids into the reactor.

However, one such tube 23 made of PEEK can also be used to aspirate liquid from the reactor. For this purpose it must reach the bottom. This is accomplished by moving tube support 24 down by means of pneumatic actuator 25. When it is shifted to a lower position, the tube extends to the bottom of the reactor 9, as shown in the drawing. To avoid possible contamination of reaction mixture that may result from prolonged contact with PEEK tube 23 and possible blockage of said tube by dry residue from evaporation, the tube is retracted during other stages of the process by moving actuator 24 to its upper position which positions the end of tube 23 approximately 30 mm off the bottom.

Reactor cap 21 is attached to pivoting frame 27. Parts 17-25, attached to frame 27, comprise a reactor assembly which pivots around center of rotation 30 by force of pneumatic actuator 26. In the drawing, the reactor assembly is shown in its inclined position. When in this position, the reactor axis is at approximately 50° to vertical and the reactor side wall is close to horizontal. In this position, the area of reactor wall covered by the liquid is maximized. However, when in an inclined position, complete aspiration of liquid from reactor 9 via tube 23 is not possible, because the tube opening cannot be placed at the lowest point of the reactor. To achieve complete extraction of liquid, the reactor must be positioned so that its axis is close to vertical. This can be accomplished by first lowering heating bath 16 and then retracting the plunger of actuator 26 which causes pivot frame 27 to rotate counterclockwise approximately 50° and brings the reactor flask to a substantially vertical position.

Electric gear motor 29 (24VDC 82 RPM, Pittman, Harlesville, Pa.) is attached to a pivot frame so that its position relative to frame 27 is fixed. Motor 29 is used to rotate the reactor. For this purpose, a pulley is attached to the shaft of motor 27 and timing belt 28 connects the pulley to rotor 17. Motor 27 turns at 86 rpm causing reactor 9 to rotate at 60-80 rpm, adjustable by selection of pulley size.

When, for example, a 10 ml cone-bottom pear-shaped flask is placed at 50° from the vertical as shown in FIG. 3, filled with 1 ml of liquid and revolved around its axis, the wetted wall area of the flask is approximately 15 cm², as determined by calculation using 3D modeling software (Desault Systemes Solidworks Corp., Concord, Mass.). Thus is five times greater area than is normally covered by liquid in a traditional stationary flat-bottom or round bottom reactor with an inside diameter of 2 cm. Use of a 25 ml flask would increase this area to 30 cm² making it ten times greater than that of a traditional reactor. As discussed below, this surface area over which the radioactive material is spread during and after a drying step and during reaction and a subsequent second evaporation is directly related to the system suitability for efficient processing of large amounts of radioactive material.

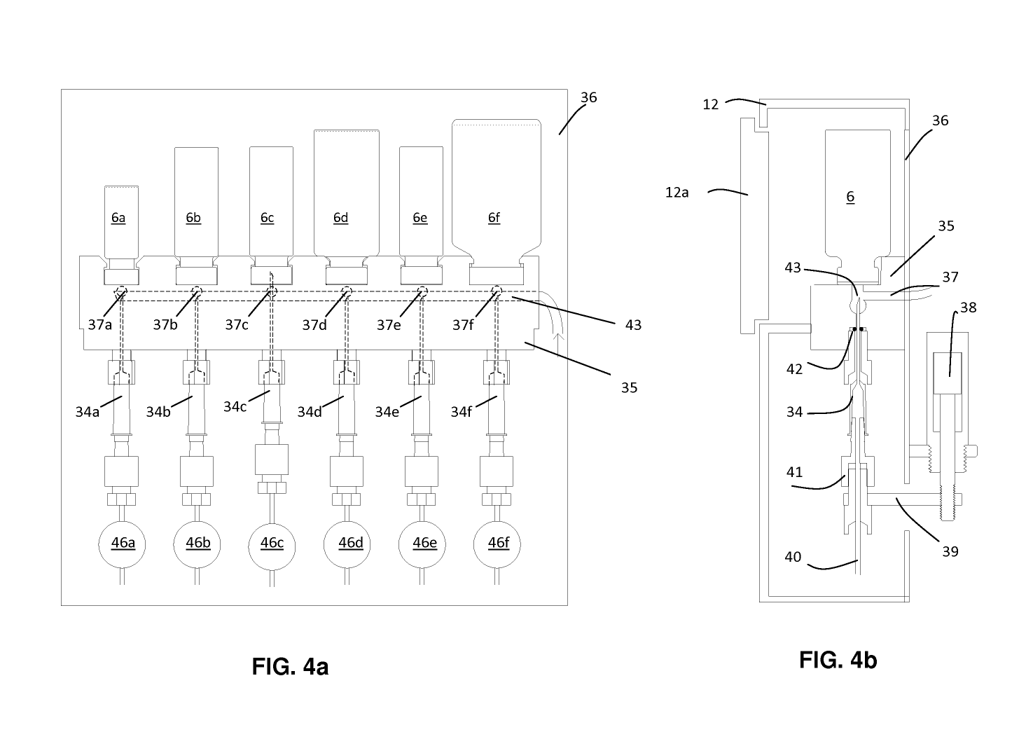

To safely introduce necessary reagents into reactor 9, a separate reagent module is constructed and placed within its own clean air enclosure outside of the lead shielding. As shown in FIG. 4a, components of a reagent module are mounted on a vertical supporting plate 36 made of stainless steel. Reagent storage containers which are standard sealed crimp-top serum vials 6a-6f are manually placed on the support 35 designed to accept vials of different size from 1 ml to 30 ml capacity. Vial seals remain intact until penetrated by needles 34a-34f as illustrated by needle 34c. Channels 37a-37f connect externally to a source of clean air. Channel 43 intersects all needle cavities and is used for cleaning, as discussed below. When all containers are introduced, the enclosure door is closed and a clean air environment within the enclosure is established. Fluid transport to and from needles 34a-34f is controlled by valves 46a-46f.

FIG. 4b illustrates the reagent module in an environmental enclosure 12 wherein the supporting plate 36 forms the back and access to a typical vial 6 is through a transparent (for convenience) door 12a that makes a seal to the enclosure. The enclosure makes a seal to block 35, but vial 6 is a lose fit to block 35. As shown in a cross-sectional view of a reagent module in FIG. 4b, the needle 34 for each reagent vial 6 is attached to fitting 41 supported by moving plate 39 which can be moved up and down by pneumatic actuator 38. The lower portion of the enclosure containing the needle moving mechanism 39 & 41 is isolated from the upper part containing vials 6 and filled with clean air. The lower part is not sealed from the environment outside. To reduce the risk of contamination, the needle 34 remains in its lower position and reagent container 6 seal remains intact until after all manual manipulations within the clean air enclosure are completed, the door of the enclosure is closed, and a clean air environment within the enclosure is established.

When lifted up needle 34 penetrates the septum of vial 6 and reagent contained therein can flow though tube 40 into the reactor 9. When reagent transfer is complete, the needle is retracted to its lower position to enable easy removal and replacement of the reagent vial. Alternatively, the same can be accomplished by using a stationary needle and moving the vial holder to cause the needle to penetrate the septum.

To reduce risk of contamination, the following method of cleaning and sanitizing the needles is employed: Each needle, when retracted, is situated within channel 43 which is filled and flushed with a cleaning and sterilizing agent such as sterile alcohol supplied by means of a peristaltic pump through an external connection to one end of the channel as shown in FIG. 4a. Cleaned needles and the channel can then be dried in a flow of clean filtered air supplied through channel 37, thus rendering the apparatus cleaned, sanitized and ready for re-use. To prevent leaks of cleaning agent as well as contamination from the bottom, the opening through which needle 34 is inserted is sealed with O-ring 42. This clean-in-place (CIP) and sterilize-in-place (SIP) procedure is utilized each time reagent vials are replaced and a new batch of the product is manufactured.

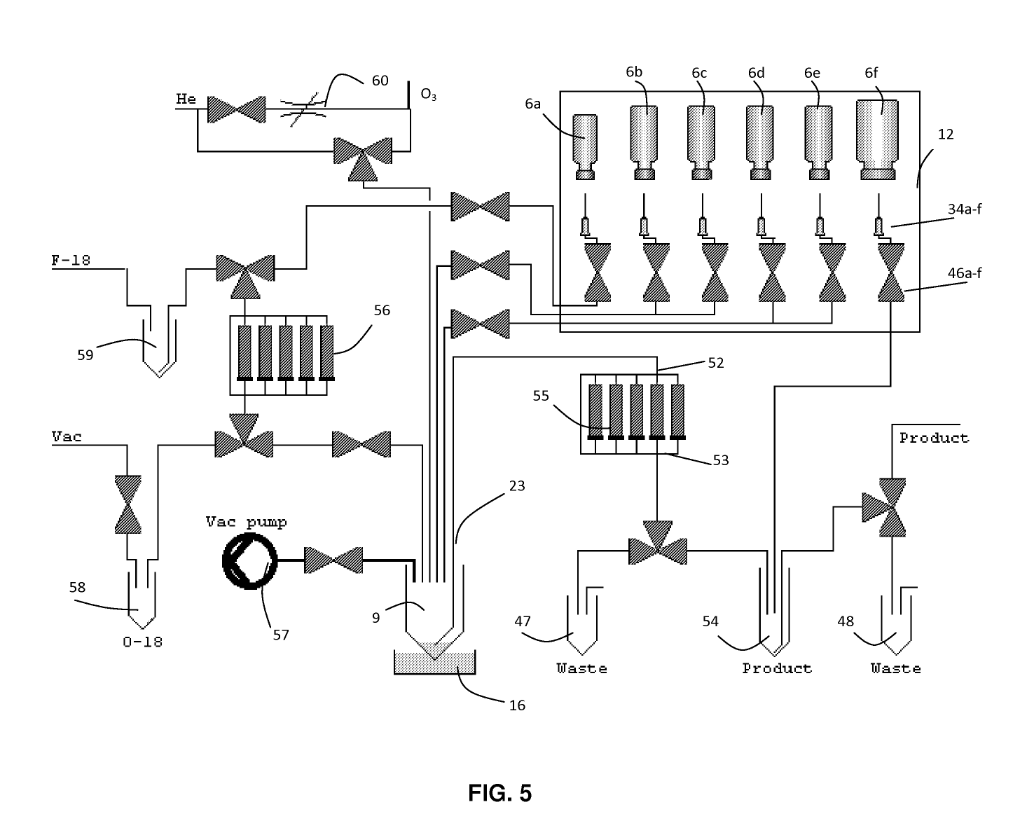

Referring to FIG. 5, which is a schematic representation of the processing and reagent modules, in conjunction with FIGS. 4a and 4b, a method to further reduce the risk of contamination of the entire reactor module and reagent enclosure will be explained. Sterilization is accomplished using an appropriate gaseous sterilant such as vaporized hydrogen peroxide or, preferably, ozone. Ozone can be produced from oxygen gas at a flow rate of 0.5 Lpm using an in-line generator, for example, Ozone Generator Model 1000 (Jelight Company Inc., Irvine, Calif.) which produces over a 6000 ppm ozone concentration. This is directed into the gas inlet port, designated O₃, downstream from metering valve 60 (FIG. 5, top), where it is diluted with a stream of helium (He) to approximately 3000 ppm ozone concentration, sufficient to achieve partial (i.e., absolute is generally not obtainable) sterilization. Sterilant mixture fills the reactor 9 and is directed through associated valves and tubes to exit through the needles 34c-34f to fill channel 43. With the feed for channels 37 and 43 blocked off, sterilant is forced past the serum vials 6 into the chamber 12. With the appropriate valves open, all processing module flasks and tubing coming in contact with the product and reagents can be partially sterilized after a 1-4 hour exposure to ozone. Excess sterilant can be safely released into room air where it is diluted to less than a 1 ppm level. The residue of sterilizing agent is removed from the reactor module by vacuum pump 57 in a stream of inert helium (He) gas. Preferably, proper cleaning, sanitization, sterilization, and line clearance procedures are performed before allowing new batch processing to begin. However, if time does not permit, sterilization at the beginning of a work day would still be beneficial. Sterilization may be limited to just reactor 9 or just reagent enclosure 12 with, preferably, door 12a closed during the procedure.

Referring now to FIG. 4a, complete isolation of all critical surfaces is impossible because reagents must be introduced into the system and, consequently, certain operations, such as penetrating reagent vial seals 6 with a needle 34, require that the inserted part of the needle is outside the sealed vial. Therefore, to even further reduce contamination, the invention provides a controlled environment surrounding the area where such operations take place to exclude contaminants, such as dust and bacterium, by supplying clean air filtered through a 0.2 micron filter. This clean air is directed via openings 37 behind channel 43 and sweeps across the area where septa are penetrated by the needles. Air flow and particulate content are controlled to preferably satisfy at least ISO class 7 clean air requirements, i.e. laminar flow at 90 fpm and less than 100 particles 0.5 micron and larger per cu. ft. An automatic interlock is provided to prevent movement of reagent needles 34 while the door 12a is open. A sampling port is provided for monitoring air quality in the compartment. The measures described herein make it possible to comply with GMP requirements without having a clean room.

The above described reactor module and reagent module with a multitude of standard solenoid valves (LFRX and LFVA type supplied by Lee Co., Westbrook, Conn.), and other standard components comprise the system schematically represented in FIG. 5. Pressure and temperature are monitored by means of a PX603 pressure transducer and a Pt100 RTD probe (supplied by Omega Scientific, Stamford, Conn.). Vessels used for collecting liquids are screw cap V-bottom shaped vials of suitable volume (supplied by Wheaton Industries, Millville, N.J.). Reagent storage vials are standard serum crimp-seal vials (available from Wheaton Industries, Millville, N.J.). Vacuum connections are ¼″ OD silicon polymer (Tygon®) tubing, the vacuum shut off valve is pneumatically actuated model MT442 (supplied by BECO Mfg, Laguna Hills, Calif.). All other fluid connections are via 1/16″ OD PEEK tubing. Liquids are typically transferred by application of helium pressure of 100 kPa and/or vacuum or by means of a miniature pump.

The system is controlled by a 96-channel Programmable Logic Controller (PLC) with an 8″ TFT touch screen interface (supplied by EZAutomation, Bettendorf, Iowa). The automated controller with the included operator interface is designed to control and record all relevant in-process parameters and document process execution including all deviations, to comply with GMP requirements.

### Prophetic Example

Referring to FIG. 5 (wherein all flasks are physically sealed), FDG synthesis can be carried out as follows. Incoming F-18 solution from the cyclotron is deposited into V-bottom vial 59 and then passed through one of five anion exchange cartridges 56 containing 40-130 mg of alkylamine-modified, 37-55 μm, silica-based, anion exchange sorbent, (Sep-Pack® QMA, supplied by Waters Technologies Corp., Milford, Mass.). To direct flow through a selected cartridge two pneumatically actuated six-position flow selector valves, not shown in the diagram, (Cheminert C25, supplied by VICI, Houston, Tex.) are used. Only five positions are used to select cartridges, one is reserved for bypass which is useful for a clean-in-place (CIP) process. Solvents used in CIP procedure are collected in waste flasks 47 and 48.

Anion exchange cartridge 56 retains F-18 fluoride while the O-18 water is directed into the storage container 58 to be later recycled and reused. After removing all water from the cartridge, 1 ml of 60% acetonitrile in water, containing 20 mg of phase transfer catalyst (Kryptofix® 222 supplied by Merck, Whitehouse Station, N.J.) and 4 mg of potassium bicarbonate, contained in the 2-ml serum vial 6a is passed through cartridge 56 and collected in reactor 9.

Unlike conventional processing, the reactor is continuously rotating and is tilted to an inclined position at about 50 degrees to vertical, after which heating bath 16 is lifted up and heater (31 in FIG. 3) turned on which causes heating bath 16 temperature to gradually increase to 100° C. Vacuum pump 57 (dual stage diaphragm pump model N84.4 supplied by KNF Neuberger, Trenton, N.J.) is turned on to evacuate air from reactor 9 and facilitate evaporation of water. At the same time, a stream of helium is directed into the reactor to remove water vapor. Gas flow rate is regulated by manual metering valve 45 (model FC10AV supplied by VICI, Houston, Tex.) to achieve pressure inside reactor 9 in a range of 20-50 kPa. Reactor 9 is revolved at 60-80 rpm. Complete evaporation of the reaction mixture is achieved in 2-3 min, at which time 1 ml of dry acetonitrile may be optionally added from 2-ml serum vial 6b and once again evaporated for 1-2 min to completely remove any residue of water from the reaction mixture.

Helium flow is then stopped, the residue is dried in vacuum at or below 1 kPa for 1 min, the heating bath 16 temperature is adjusted to 80° C., and the vacuum pump 57 is switched off.

Then, a 1 ml solution of 20-30 mg of precursor (1,3,4,6-tetra-O-acetyl-2-O-trifluoro-methanesulfonylbeta-D-mannopyranose, supplied by ABX, Radeberg, Germany) in dry acetonitrile is added to the reactor 9 from serum vial 6c and the mixture is allowed to react for 2-3 min while reactor 9 continues to revolve to facilitate mixing.

After the reaction period is complete, the vacuum pump and helium flow are once again switched on and the acetonitrile is evaporated for 1-2 min.

Next, 5 ml of 0.2N sodium hydroxide solution is added to the reactor from the 10 ml serum vial 6e and heating bath 16 temperature is adjusted to 50° C. Reactor 9 is revolved for 3-5 min to complete the hydrolysis. At the end of this period, heating bath 16 is lowered and reactor 6 tilted into the upright position. Moveable tube 23 is lowered to the bottom of the reactor and reaction product mixture is aspirated from the reactor and passed through one of five purification columns 55 and collected in product storage flask 54. To direct flow through a selected column, a pair of pneumatically actuated six-position flow selector valves (not explicitly illustrated in the diagram), at locations 52 and 53, (Cheminert C25, supplied by VICI, Houston, Tex.) are used. As before, only five positions are used to select columns, one is reserved for bypass which is useful for a clean-in-place process. Each of the five columns 55 is comprised of three cartridges, containing, in order from top to bottom: 600 mg of styrene-divinylbenzene base modified with sulfonic acid in hydrogen form (MaxiClean™ SCX supplied by Alltech, Deerfield Ill.), 1700 mg of basic aluminum oxide (Sep-Pack™ supplied by Waters, Milford Mass.), and 360 mg of silica gel modified with octadecanoic acid (Sep-Pack™ C18 supplied by Waters, Milford, Mass.).

To remove any residue of useful product from the purification cartridges, 5 ml of water is added to reactor 9 from serum vial 6f and then passed through the same column 55 and added to product vessel 54. To adjust the concentration of FDG in the final product, typically 500-1000 mCi/ml is desired, the required volume of a 0.9% sodium chloride solution is added to product vessel 54 from serum bottle 51. This completes an FDG synthesis process which takes less than 20 min.

Although not being bound by any particular theory, this inventor believes that reduction in yield observed when high amounts of radioactive material are processed is due to the fact that positrons emitted by F18 isotope may cause decomposition of any or all of the product, intermediates, reagents, solvents or catalysts involved, and that such adverse effect is most severe within close proximity to the place where said positrons originate and is especially aggravated at such times when the reaction mixture is most concentrated, for example, immediately after evaporation. Autoradiolysis may be reduced by spreading the reaction mixture as it evaporates over a large area to form a residual layer with thickness substantially less than the range a positron normally travels which is 0.5 mm (Bai et al., Nuclear Science Symposium Conference Record, 2005 IEEE Vol: 5 pp 2686-2689. (2005)). This should allow most positron particles to escape from the reaction mixture before they cause damage to the reactants. Such theory, although not taught in the prior FDG art, is consistent with fundamental knowledge of positrons interaction with matter

One method of spreading liquid over a large area is rotating a reaction flask around a substantially horizontal axis, thus making the liquid wet the walls of the vessel. As the liquid evaporates, the residue will form a relatively thin film over the large area. Other possible methods of spreading evaporation residue over a large area include, for example, spreading the liquid over an essentially flat bottom of the reactor vessel by gravity or by agitation, circulating the liquid by a pump and allowing it to flow along an inclined or a vertical surface, pouring liquid onto a spinning disk or placing it into a cylindrical flask spinning around a vertical axis with an angular speed sufficient to spread the liquid over the walls by centrifugal force.

Regardless of the method by which the reaction mixture is spread, a large reactor surface area, in one embodiment of this invention of about 30 cm², would allow efficient production of over 10 Ci of FDG in each batch with minimal exposure of the operator to ionizing radiation because no access to activated components will be required to reload reagents. At the same time, product contamination is minimized and compliance with GMP requirements is easily assured even without having the apparatus situated within a clean room.

1. A method for automated synthesis of a fluorine F18-labeled FDG compound comprising the steps of:
a) providing at least one rotating reactor having a rotation axis and having a motorized mechanism to rotate said reactor and another motorized mechanism to tilt said reactor rotation axis from a substantially vertical to an inclined angle, both under automatic control; and
b) introducing F-18 radioisotope in the form of a fluoride solution in water and at least one reactant into said reactor to form a reaction mixture and perform synthesis via a nucleophilic substitution reaction;
c) during a substantial part of the synthesis, tilting said reactor to and inclined position and rotating said reactor about its axis, so that said reaction mixture is essentially spread over the reactor walls; and
d) placing said reactor in a near vertical position to allow essentially complete removal of the reaction products.

2. The method of claim 1 wherein said reaction mixture volume is less than about 1 ml.

3. The method of claim 2 wherein said reactor has a volume of at least 10 ml.

4. The method of claim 1 wherein said reactor has a volume of at least 25 ml.

5. The method of claim 1 wherein said reactor is a cone-bottom pear shaped flask.

6. The method of claim 1 wherein reaction products are extracted by tilting said reactor to a substantially vertical position, inserting an extraction tube, extracting said products, and retracting said extraction tube in preparation for a succeeding batch.

7. A method for introducing reagents into a reactor apparatus inside a shielded enclosure for synthesizing a fluorine F18-labeled radiopharmaceutical which includes the following steps:
a) providing a separate environmentally protected reagent enclosure placed outside of said shielded enclosure containing said reactor apparatus;
b) opening said enclosure and manually placing at least one single-use sealed container containing at least one reagent into said reagent enclosure in a manner that said container seal remains intact until a further step;
c) closing said reagent enclosure and purging with clean filtered air to remove contamination; and
d) automatically penetrating said reagent container seal to cause the reagent to be deposited into said reactor apparatus by application of vacuum therein.

8. The method in claim 7 further comprising sterilizing-in-place said reactor apparatus by gaseous sterilant.

9. The method in claim 8 wherein said sterilant is ozone.

10. The method in claim 9 wherein said ozone is produced by an in-line ozone generator.

11. The method in claim 8 wherein said sterilant is vaporized hydrogen peroxide.

12. The method in claim 7 wherein air within the reagent enclosure in step “c” is ISO class 7 or better.

13. The method in claim 7 wherein the reagent enclosure is sterilized-in-place by a gaseous sterilant.

14. The method in claim 13 wherein said sterilant is ozone.

15. The method in claim 13 wherein said sterilant is vaporized hydrogen peroxide.

16. A method for automated synthesis of a fluorine F18-labeled FDG compound comprising the steps of:
a) providing at least one rotating reactor having a rotation axis and having a motorized mechanism to rotate said reactor and another motorized mechanism to tilt said reactor rotation axis from a substantially vertical to an inclined angle, both under automatic control; and
b) introducing F-18 radioisotope in the form of a fluoride solution and at least one reactant into said reactor to form a reaction mixture and perform synthesis via a nucleophilic substitution reaction;
c) during a substantial part of the synthesis, tilting said reactor to an inclined position and rotating said reactor about its axis, so that said reaction mixture is essentially spread over a wetted surface area (in cm²) of at least 15 times the volume (in ml) of said reaction mixture; and
d) placing said reactor in a near vertical position to allow essentially complete removal of the reaction products.

17. The method in claim 1 where said reaction mixture volume is about 1 ml or less and said wetted surface area is at least 15 cm².

18. The method in claim 1 where said reaction mixture volume is about 1 ml or less and said wetted surface area is at least 30 cm².

19. The method of claim 1 wherein said reactor is a cone-bottom pear shaped flask.

20. The method of claim 1 wherein reaction products are extracted by tilting said reactor to a substantially vertical position, inserting an extraction tube, extracting said products, and retracting said extraction tube in preparation for a succeeding batch.
